(19) United States
(12) Patent Application Publication
(10) Pub. No.: US 2026/0060804 A1
(43) Pub. Date: Mar. 5, 2026

(54) ROTATIONAL ALIGNMENT OF MEDICAL IMPLANT

(71) Applicant: BOSTON SCIENTIFIC SCIMED, INC., MAPLE GROVE, MN (US)

(72) Inventors: Richard Kiely, Galway (IE); David Bishop, Carrickfergus (GB)

(73) Assignee: BOSTON SCIENTIFIC SCIMED, INC., MAPLE GROVE, MN (US)

(21) Appl. No.: 19/383,090

(22) Filed: Nov. 7, 2025

Related U.S. Application Data
(63) Continuation of application No. 17/716,291, filed on Apr. 8, 2022, now Pat. No. 12,485,008.
(60) Provisional application No. 63/173,097, filed on Apr. 9, 2021.

(51) Int. Cl. A61F 2/24; A61F 2/95; A61M 25/09

(52) U.S. Cl. CPC A61F 2/2436; A61F 2/2418; A61F 2/9517; A61M 25/09

(57) ABSTRACT

A system for delivering a replacement heart valve implant may include the implant including an expandable framework and leaflets coupled to the framework, and a delivery device including a handle and an elongate catheter extending from the handle. The catheter includes an outer sheath and an inner shaft movably disposed within the outer sheath. The implant is releasably coupled to the inner shaft. The inner shaft includes an orientation lumen extending longitudinally within the inner shaft, the orientation lumen being offset from a central guidewire lumen of the inner shaft. The delivery device includes an orientation mandrel disposed within the orientation lumen, the orientation mandrel extending distally from the handle to a distal end proximate a closed distal end of the orientation lumen. The orientation mandrel is operably coupled to an orientation mechanism of the handle configured to apply compressive force or tensile force to the orientation mandrel.

### CROSS-REFERENCE TO RELATED APPLICATIONS

This application is a continuation of U.S. Application No., 17/716,291, filed April 8, 2022, which claims the benefit of priority of U.S. Provisional Application No. 63/173,097 filed April 9, 2021, the entire disclosure of which is hereby incorporated by reference.

### TECHNICAL FIELD

The present disclosure pertains to medical devices, and methods for manufacturing and/or using medical devices. More particularly, the present disclosure pertains to a system for delivering a medical implant.

### BACKGROUND

A wide variety of intracorporeal medical devices have been developed for medical use, for example, intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

In a first example, a system for delivering a replacement heart valve implant to a native heart valve of a patient may comprise a replacement heart valve implant including an expandable framework and a plurality of leaflets coupled to the expandable framework at commissural posts of the expandable framework; and a delivery device including a proximal handle and an elongate catheter extending distally from the proximal handle, wherein the elongate catheter includes an outer sheath and an inner shaft movably disposed within a lumen of the outer sheath. The replacement heart valve implant may be releasably coupled to a distal portion of the inner shaft in a delivery configuration. The inner shaft may include a central guidewire lumen extending longitudinally therethrough. The inner shaft may include an orientation lumen extending longitudinally within the inner shaft, the orientation lumen being laterally offset from the central guidewire lumen and having a closed distal end. The delivery device may include an orientation mandrel slidably disposed within the orientation lumen, the orientation mandrel extending distally from the proximal handle within the orientation lumen to a distal end proximate the closed distal end of the orientation lumen. The orientation mandrel may be operably coupled to an orientation mechanism of the proximal handle, the orientation mechanism being configured to move the orientation mandrel axially relative to the inner shaft.

In addition or alternatively to any example disclosed herein, a distal portion of the elongate catheter includes a pre-formed curve configured to conform to an aortic arch of the patient, the pre-formed curve defining an arch of the elongate catheter having an inside bend and an outside bend disposed opposite the inside bend relative to the central guidewire lumen.

In addition or alternatively to any example disclosed herein, the orientation mandrel is disposed along the inside bend of the arch of the elongate catheter in a first configuration.

In addition or alternatively to any example disclosed herein, the orientation mandrel is disposed along the outside bend of the arch of the elongate catheter in a second configuration.

In addition or alternatively to any example disclosed herein, the orientation mechanism is configured to advance the orientation mandrel distally relative to the inner shaft.

In addition or alternatively to any example disclosed herein, distal advancement of the distal end of the orientation mandrel against the closed distal end of the orientation lumen causes compressive force to build up within the orientation mandrel.

In addition or alternatively to any example disclosed herein, the compressive force shifts the elongate catheter from the first configuration to the second configuration.

In addition or alternatively to any example disclosed herein, a system for delivering a replacement heart valve implant to a native heart valve of a patient may comprise a replacement heart valve implant including an expandable framework and a plurality of leaflets coupled to the expandable framework at commissural posts of the expandable framework; and a delivery device including a proximal handle and an elongate catheter extending distally from the proximal handle, wherein the elongate catheter includes an outer sheath and an inner shaft movably disposed within a lumen of the outer sheath. The replacement heart valve implant may be releasably coupled to a distal portion of the inner shaft in a delivery configuration. The inner shaft may include a central guidewire lumen extending longitudinally therethrough. The delivery device may include a first orientation mandrel attached to an outside surface of the inner shaft, the first orientation mandrel extending distally from the proximal handle to a first distal end fixedly attached to the inner shaft proximate a distal end of the inner shaft. The first orientation mandrel may be operably coupled to an orientation mechanism of the proximal handle, the orientation mechanism being configured to apply compressive force or tensile force to the first orientation mandrel.

In addition or alternatively to any example disclosed herein, a distal portion of the elongate catheter includes a pre-formed curve configured to conform to an aortic arch of the patient, the pre-formed curve defining an arch of the elongate catheter having an inside bend and an outside bend disposed opposite the inside bend relative to the central guidewire lumen.

In addition or alternatively to any example disclosed herein, the first orientation mandrel may be disposed along the inside bend of the arch of the elongate catheter in a first configuration. The first orientation mandrel may be disposed along the outside bend of the arch of the elongate catheter in a second configuration.

In addition or alternatively to any example disclosed herein, applying compressive force against the first distal end fixedly attached to the inner shaft causes rotation of the inner shaft in a first direction to shift the elongate catheter from the first configuration toward the second configuration.

In addition or alternatively to any example disclosed herein, applying tensile force to the first distal end fixedly attached to the inner shaft causes rotation of the inner shaft in a second direction to shift the elongate catheter from the first configuration toward the second configuration.

In addition or alternatively to any example disclosed herein, the delivery device includes a second orientation mandrel attached to an outside surface of the inner shaft opposite the first orientation mandrel relative to the central guidewire lumen, the second orientation mandrel extending distally from the proximal handle to a second distal end fixedly attached to the inner shaft proximate the distal end of the inner shaft. The second orientation mandrel may be operably coupled to the orientation mechanism of the proximal handle, the orientation mechanism being configured to apply compressive force or tensile force to the second orientation mandrel.

In addition or alternatively to any example disclosed herein, the second orientation mandrel may be disposed along the outside bend of the arch of the elongate catheter in the first configuration. The second orientation mandrel may be disposed along the inside bend of the arch of the elongate catheter in the second configuration.

In addition or alternatively to any example disclosed herein, applying compressive force against the second distal end fixedly attached to the inner shaft causes rotation of the inner shaft in a second direction opposite the first direction to shift the elongate catheter from the first configuration toward the second configuration.

In addition or alternatively to any example disclosed herein, a method of positioning a replacement heart valve implant within a native heart valve of a patient may comprise:

advancing a system percutaneously to a position adjacent the native heart valve, the system comprising a replacement heart valve implant including an expandable framework and a plurality of leaflets coupled to the expandable framework at commissural posts of the expandable framework, and a delivery device including a proximal handle and an elongate catheter extending distally from the proximal handle, wherein the elongate catheter includes an outer sheath and an inner shaft movably disposed within a lumen of the outer sheath, wherein the replacement heart valve implant is releasably coupled to a distal portion of the inner shaft in a delivery configuration, wherein the inner shaft includes an orientation lumen extending longitudinally within the inner shaft, the orientation lumen being laterally offset from a central guidewire lumen and having a closed distal end;

deploying the replacement heart valve implant within the native heart valve; and aligning the commissural posts of the replacement heart valve implant with commissures of the native heart valve by rotating the replacement heart valve implant relative to the outer sheath.

In addition or alternatively to any example disclosed herein, the delivery device may include an orientation mandrel disposed within the orientation lumen, the orientation mandrel extending distally from the proximal handle within the orientation lumen to a distal end proximate the closed distal end of the orientation lumen. The orientation mandrel may be operably coupled to an orientation mechanism of the proximal handle, the orientation mechanism being configured to apply compressive force or tensile force to the orientation mandrel.

In addition or alternatively to any example disclosed herein, the method may further comprise applying compressive force or tensile force to the orientation mandrel using the orientation mechanism to rotate the replacement heart valve implant relative to the outer sheath.

In addition or alternatively to any example disclosed herein, prior to applying compressive force or tensile force to the orientation mandrel, the orientation mandrel is disposed along an inside bend of the elongate catheter within an aortic arch of the patient.

In addition or alternatively to any example disclosed herein, after applying compressive force or tensile force to the orientation mandrel, the orientation mandrel is disposed along an outside bend of the elongate catheter within the aortic arch of the patient.

The above summary of some embodiments, aspects, and/or examples is not intended to describe each disclosed embodiment or every implementation of the present disclosure. The Figures and Detailed Description more particularly exemplify aspects of these embodiments.

### BRIEF DESCRIPTION OF THE DRAWINGS

The disclosure may be more completely understood in consideration of the following detailed description in connection with the accompanying drawings, in which:

FIG. 1 schematically illustrates an example configuration of a heart;

FIG. 2 schematically illustrates an example replacement heart valve implant;

FIG. 3 schematically illustrates aspects of a system for delivering the replacement heart valve implant;

FIG. 4 is a cross-section taken at line 4–4 of FIG. 3;

FIG. 4A is an alternative cross-section taken at line 4–4 of FIG. 3;

FIG. 4B is an alternative cross-section taken at line 4–4 of FIG. 3;

FIG. 5 schematically illustrates aspects of the system for delivering the replacement heart valve implant;

FIG. 6 is a cross-section taken at line 6–6 of FIG. 5;

FIG. 6A is an alternative cross-section taken at line 4–4 of FIG. 5 corresponding to the configuration of FIG. 4A;

FIG. 6B is an alternative cross-section taken at line 4–4 of FIG. 5 corresponding to the configuration of FIG. 4B;

FIG. 7 schematically illustrates aspects of the system for delivering the replacement heart valve implant;

FIG. 8 is a cross-section taken at line 8–8 of FIG. 7;

FIG. 9 schematically illustrates aspects of an alternative embodiment of the system for delivering the replacement heart valve implant;

FIG. 10 is a cross-section taken at line 10–10 of FIG. 9;

FIG. 11 schematically illustrates aspects of an alternative embodiment of the system for delivering the replacement heart valve implant;

FIG. 12 is a cross-section taken at line 12–12 of FIG. 11; and

FIG. 13 schematically illustrates aspects of an alternative embodiment of the system for delivering the replacement heart valve implant;

FIG. 14 is a cross-section taken at line 14–14 of FIG. 13; and

FIGS. 15-17 illustrate aspects of a method of delivering the replacement heart valve implant to a native heart valve.

While aspects of the disclosure are amenable to various modifications and alternative forms, examples are shown in the drawings and described herein. It should be understood, however, that the intention is not to limit aspects of the disclosure to the particular embodiments described. On the contrary, the disclosure shall cover all modifications, equivalents, and alternatives falling within the spirit and scope thereof.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified .

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

The terms “transaortic valve implantation” and “transcatheter aortic valve implantation” may be used interchangeably and may each be referred to using the acronym “TAVI”. The terms “transaortic valve replacement” and “transcatheter aortic valve replacement” may be used interchangeably and may each be referred to using the acronym “TAVR”.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact the cardiovascular system are prevalent throughout the world. Traditionally, treatment of the cardiovascular system was often conducted by directly accessing the impacted part of the system. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed, for example, where a blocked coronary artery could be accessed and treated via a percutaneous catheter (e.g., angioplasty). Such therapies have gained wide acceptance among patients and clinicians.

Some mammalian hearts (e.g., human, etc.) include four heart valves: a tricuspid valve, a pulmonary valve, an aortic valve, and a mitral valve. Some relatively common medical conditions may include or be the result of inefficiency, ineffectiveness, or complete failure of one or more of the valves within the heart. Treatment of defective heart valves poses other challenges in that the treatment often requires the repair or outright replacement of the defective valve. Such therapies may be highly invasive to the patient. Disclosed herein are systems, devices, and/or methods that may be used within a portion of the cardiovascular system in order to diagnose, treat, and/or repair the system, for example during and/or in conjunction with a TAVI or TAVR procedure, or in place of a TAVI or TAVR procedure in patients not suitable for such. At least some of the systems, devices, and/or methods disclosed herein may be used percutaneously and, thus, may be much less invasive to the patient, although other surgical methods and approaches may also be used. The systems, devices, and/or methods disclosed herein may also provide a number of additional desirable features and benefits as described in more detail below. For the purpose of this disclosure, the discussion below is directed toward the treatment of a native aortic valve and will be so described in the interest of brevity. This, however, is not intended to be limiting as the skilled person will recognize that the following discussion may also apply to a mitral valve or another heart valve with no or minimal changes to the structure and/or scope of the disclosure. Similarly, the medical devices disclosed herein may have applications and uses in other portions of a patient’s anatomy, such as but not limited to, arteries, veins, and/or other body lumens.

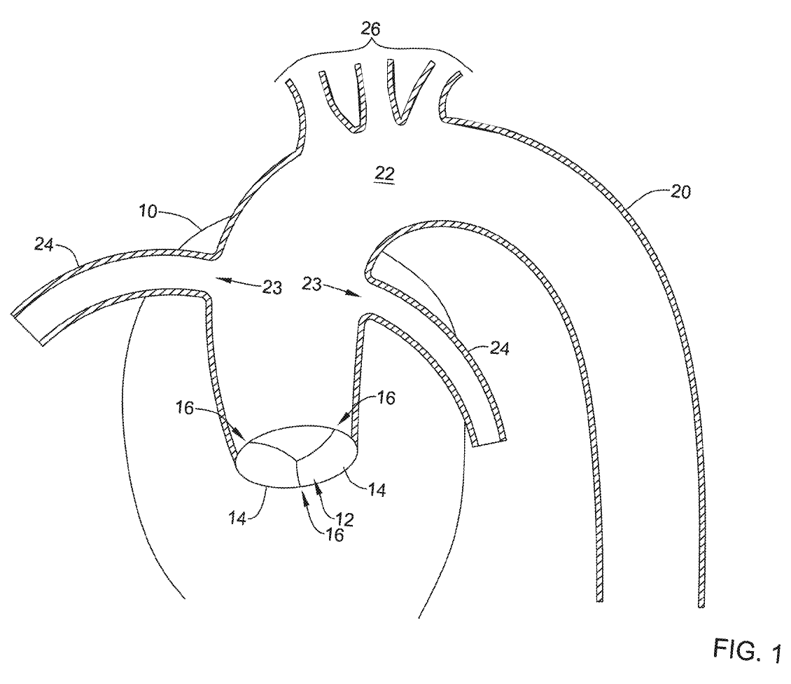

FIG. 1 illustrates a schematic partial cut-away view of a portion of a patient’s heart 10 including a native heart valve 12 (e.g., the aortic valve) having a plurality of valve leaflets 14, and certain connected vasculature, such as the aorta 20 connected to the aortic valve 12 of the heart 10 by the aortic arch 22, the coronary arteries 24, the ostia 23 of the coronary arteries 24, and other large arteries 26 (e.g., subclavian arteries, carotid arteries, brachiocephalic artery) that extend from the aortic arch 22 to important internal organs. The plurality of valve leaflets 14 may meet and/or come together at commissures 16 of the native heart valve 12 (e.g., the aortic valve). As mentioned above, for the purpose of this disclosure, the discussion below is directed toward use in the native heart valve 12 (e.g., the aortic valve) and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

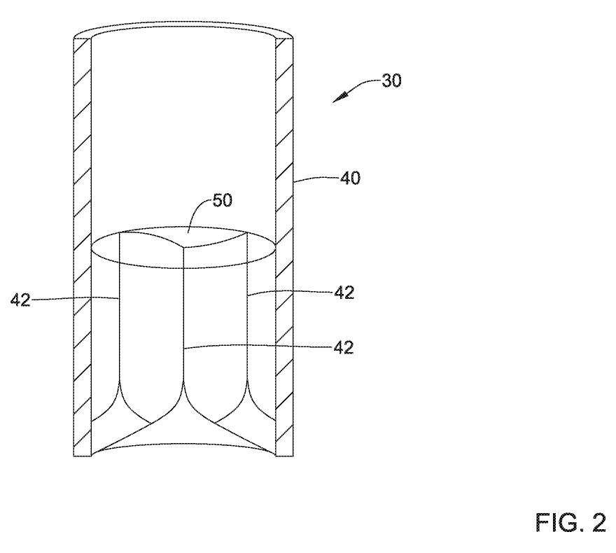

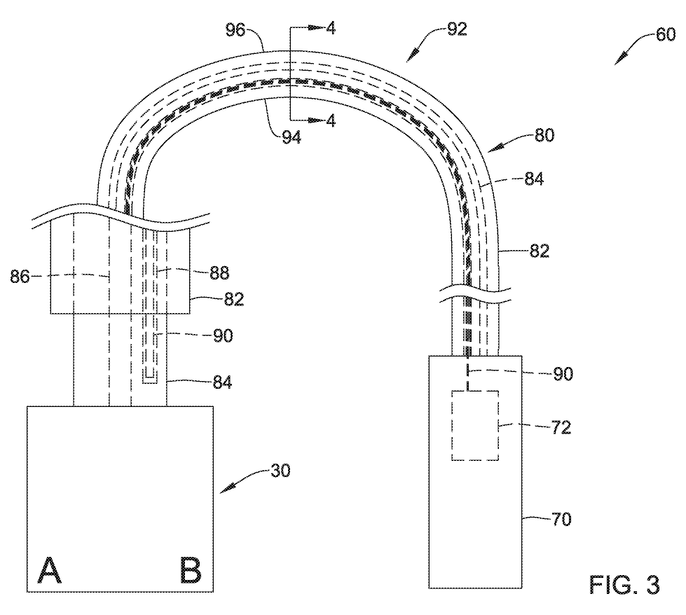

FIG. 2 illustrates a schematic example of a replacement heart valve implant 30 configured to be disposed within the native heart valve 12 (e.g., the aortic valve) of a patient and/or the patient’s heart 10. The replacement heart valve implant 30 is shown in partial cross-section. The replacement heart valve implant 30 may include an expandable framework 40 and a plurality of leaflets 50 coupled to the expandable framework 40 at commissural posts 42 of the expandable framework 40. In some embodiments, the replacement heart valve implant 30 and/or the expandable framework 40 may be reversibly actuatable between a delivery configuration and a deployed configuration, wherein in the delivery configuration the expandable framework 40 is axially elongated and/or radially collapsed and in the deployed configuration the expandable framework 40 is axially shortened and/or radially expanded.

The expandable framework 40 may form a tubular structure defining a central longitudinal axis and a lumen extending through the expandable framework 40 from an inflow end to an outflow end. In some embodiments, the expandable framework 40 may be and/or may include an expandable stent having a plurality of struts. In some embodiments, the expandable framework 40 may be and/or include a braid formed from one or more interwoven filaments (e.g., a single filament, two filaments, etc.). In some embodiments, the expandable framework 40 may be formed from a tubular member that is cut (e.g., with a laser, etc.) to define the plurality of struts. The expandable framework 40 may include a plurality of interstices (e.g., openings) disposed in the expandable framework 40. The plurality of interstices (e.g., openings) may pass from an interior of the expandable framework 40 to an exterior of the expandable framework 40 between adjacent struts and/or filaments. In some embodiments, the expandable framework 40 may include a plurality of atraumatic inflow loops or crowns proximate the inflow end and/or a plurality of atraumatic outflow loops or crowns proximate the outflow end.

In some embodiments, the expandable framework 40 may be self-expanding. In some embodiments, the expandable framework 40 may be expanded via mechanical means, using a balloon, or other suitable methods of expansion. Other configurations are also contemplated. Some suitable but non-limiting materials for the expandable framework 40, for example metallic materials, polymeric materials, shape memory materials, etc., are described below.

In some embodiments, a seal member (not shown) may be circumferentially disposed on, about, and/or adjacent the exterior of the expandable framework 40, and as the term suggests, may help to seal the exterior of the replacement heart valve implant 30 and/or the expandable framework 40 within and/or against a target site or area of interest (e.g., the native heart valve 12) upon deployment, thereby preventing leakage around the replacement heart valve implant 30 and/or the expandable framework 40. The seal member may be disposed outside of the lumen extending through the expandable framework 40. In some embodiments, the seal member may be coupled and/or secured to the expandable framework 40 at one or more locations.

In some embodiments, the seal member may include one or more layers of polymeric material. Some suitable polymeric materials may include, but are not necessarily limited to, polycarbonate, polyurethane, polyamide, polyether block amide, polyethylene, polyethylene terephthalate, polypropylene, polyvinylchloride, polytetrafluoroethylene, polysulfone, and copolymers, blends, mixtures or combinations thereof. Other suitable polymeric materials are also contemplated, some of which are discussed below.

In some embodiments, the seal member may also include a reinforcement, a reinforcing layer, and/or one or more reinforcing members added to the polymeric material prior to curing. The reinforcement, the reinforcing layer, and/or the one or more reinforcing members may comprise a woven or nonwoven fabric and may be positioned within or between the various layers. In some embodiments, the reinforcement, the reinforcing layer, and/or the one or more reinforcing members may be positioned on a radially innermost surface or radially outermost surface of the seal member. In some embodiments, at least a portion of the seal member may be formed from a fabric material having a polymeric coating. In at least some embodiments, the seal member may be impermeable to fluids and/or blood.

In some embodiments, the seal member may include a reinforcing band coupled to the seal member and/or disposed proximate the inflow end of the expandable framework 40. In some embodiments, the reinforcing band may be integrally formed with, incorporated into, adhered to, and/or at least partially embedded within the seal member. In some embodiments, the reinforcing band may be formed from a woven or nonwoven fabric material, a textile, or other thin flexible material. The reinforcing band may provide tear resistance in the vicinity of sutures, filaments, or other attachment elements associated with components or aspects of the replacement heart valve implant 30. In some embodiments, at least one seal stitch may attach the inflow end of the seal member and/or the reinforcing band to the inflow end of the expandable framework 40.

The replacement heart valve implant 30 may include the plurality of leaflets 50 (e.g., bovine pericardial, polymeric, etc.) disposed within the lumen of the expandable framework 40. In some embodiments, the plurality of leaflets 50 may include two leaflets, three leaflets, four leaflets, etc. Other configurations are also contemplated. In some embodiments, the plurality of leaflets 50 may be coupled and/or attached to the expandable framework 40 at the commissural posts 42. In some embodiments, the commissural posts 42 may be coupled and/or attached to the expandable framework 40. In some embodiments, the commissural posts 42 may be integrally and/or monolithically formed with the expandable framework 40. The replacement heart valve implant 30 may have the same number of commissural posts as leaflets. For example, in an embodiment where the plurality of leaflets 50 includes three leaflets, the replacement heart valve implant 30 may include three commissural posts 42. Other configurations are also contemplated. The plurality of leaflets 50 may be configured to open and close within the lumen of the expandable framework 40. Free ends of the plurality of leaflets 50 may be configured to contact each other in a sealing manner at a location downstream of the inflow end when the plurality of leaflets 50 is closed.

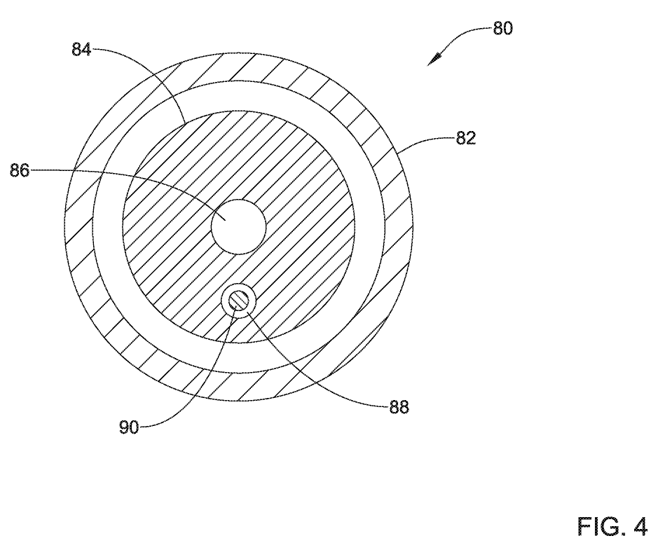

FIG. 3 illustrates aspects of a system for delivering the replacement heart valve implant 30 to the native heart valve 12 (e.g., the aortic valve) of the patient. The system may include the replacement heart valve implant 30 as described herein. For the sake of illustration and to facilitate understanding, the replacement heart valve implant 30 is labeled with reference letters A and B to indicate orientation of the replacement heart valve implant 30. The system may include a delivery device 60 including a proximal handle 70 and an elongate catheter 80 extending distally from the proximal handle 70. The elongate catheter 80 may include an outer sheath 82 having a lumen extending therethrough and an inner shaft 84 movably disposed within and/or extending longitudinally through the lumen of the outer sheath 82.

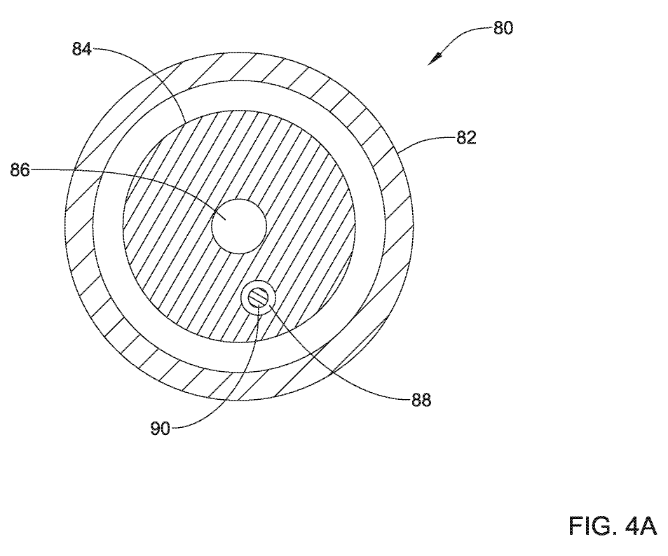

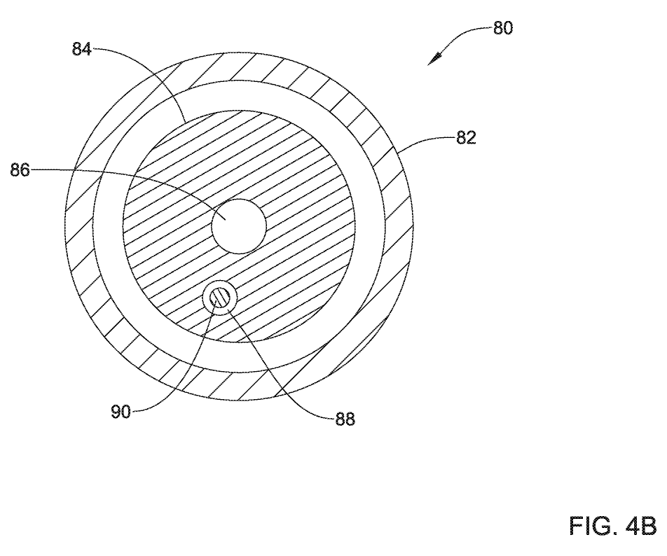

In some embodiments, the replacement heart valve implant 30 may be releasably coupled to a distal portion of the inner shaft 84 and disposed within the lumen of the outer sheath 82 in the delivery configuration during delivery of the replacement heart valve implant 30. In some embodiments, the proximal handle 70 may be disposed and/or attached at a proximal end of the delivery device 60 and may include one or more actuation means associated therewith. In some embodiments, the inner shaft 84 may include a central guidewire lumen 86 extending longitudinally therethrough. In some embodiments, the central guidewire lumen 86 may be coaxial with a central longitudinal axis of the inner shaft 84. The central guidewire lumen 86 may be configured to slidably receive and/or slidably move over a guidewire therein. In some embodiments, the inner shaft 84 may include an orientation lumen 88 (e.g., FIG. 4) extending longitudinally within the inner shaft 84. In at least some embodiments, the orientation lumen 88 may be laterally and/or radially offset from the central guidewire lumen 86 and/or the central longitudinal axis of the inner shaft 84. The orientation lumen 88 may include and/or have a closed distal end. The closed distal end of the orientation lumen 88 may be disposed proximal of a distal end of the inner shaft 84.

In some embodiments, the proximal handle 70 may include an actuation mechanism (not shown) configured to manipulate the position of the outer sheath 82 relative to the inner shaft 84 and/or the replacement heart valve implant 30, and/or to aid in the deployment of the replacement heart valve implant 30. In some embodiments, the actuation mechanism may include a rotatable knob, a slider, or other element. In some embodiments, the delivery device 60 may include a nose cone disposed at a distal end thereof. The delivery device 60 and/or the nose cone may be configured to slidably receive and/or slidably move over the guidewire (not shown). In at least some embodiments, the nose cone may have an atraumatic shape.

During delivery, the replacement heart valve implant 30 may be generally disposed in the delivery configuration within the outer sheath 82 coupled to and/or distal of the inner shaft 84. Once positioned, the outer sheath 82 may be retracted relative to the inner shaft 84 and/or the replacement heart valve implant 30 to expose the replacement heart valve implant 30. The replacement heart valve implant 30 may be actuated using the proximal handle 70 and/or the actuation mechanism in order to translate the replacement heart valve implant 30 into the deployed configuration. When the replacement heart valve implant 30 is suitably deployed within the anatomy, the replacement heart valve implant 30 may be released and/or detached from the delivery device 60 and/or the inner shaft 84 and the delivery device 60 can be removed from the vasculature, leaving the replacement heart valve implant 30 in place within the native heart valve 12 (e.g., the aortic valve) to function as, for example, a suitable replacement for the native heart valve 12.

In some embodiments, the delivery device 60 may include at least one actuator element releasably connecting the replacement heart valve implant 30 to the proximal handle 70 and/or the actuation mechanism. In some embodiments, the actuation mechanism may include an internal system disposed within the proximal handle 70. The internal system may include one or more gears, pulleys, ratchets, cams, screws, or other translation and/or locking mechanisms. In some embodiments, the at least one actuation element may be releasably connected to the commissural posts 42. In some embodiments, the at least one actuation element may be releasably connected to the expandable framework 40. Other configurations are also contemplated. In some embodiments, the at least one actuator element may extend distally from the inner shaft 84 to the replacement heart valve implant 30. In some embodiments, the at least one actuator element may be slidably disposed within and/or may extend slidably through the inner shaft 84 (in one or more unillustrated lumens, for example). In some embodiments, the at least one actuator element may be used to actuate (i.e., translate axially or longitudinally, and/or expand) the replacement heart valve implant 30 between the delivery configuration and the deployed configuration. In some embodiments, the at least one actuator element may include a plurality of actuator elements, two actuator elements, three actuator elements, four actuator elements, or another suitable or desired number of actuator elements.

In some embodiments, the delivery device 60 may include an orientation mandrel 90 disposed within the orientation lumen 88. In some embodiments, the orientation mandrel 90 may be slidably disposed within the orientation lumen 88. The orientation mandrel 90 may extend distally from the proximal handle 70 within the orientation lumen 88 to a distal end disposed and/or positioned proximate the closed distal end of the orientation lumen 88. The orientation mandrel 90 may be operably coupled to an orientation mechanism 72 of the proximal handle 70. In some embodiments, the orientation mechanism 172 may be configured to apply compressive force or tensile force to the orientation mandrel 90. In some embodiments, the orientation mechanism 72 may be configured to move at least a portion of the orientation mandrel 90 axially and/or longitudinally within the orientation lumen 88 and/or relative to the inner shaft 84. In some embodiments, the orientation mechanism 72 may include an internal system disposed within the proximal handle 70. The internal system may include one or more gears, pulleys, ratchets, cams, screws, or other translation and/or locking mechanisms capable of imparting linear movement, compressive force, and/or tensile force upon the orientation mandrel 90.

In some embodiments, a distal portion of the elongate catheter 80 may include a pre-formed curve configured to conform to the aortic arch 22 (e.g., FIG. 1) of the patient. In some embodiments, the pre-formed curve may define an arch 92 of the elongate catheter 80 having an inside bend 94 and an outside bend 96 disposed opposite the inside bend 94 relative to the inner shaft 84 and/or the central longitudinal axis of the inner shaft 84. In some embodiments, the pre-formed curve and/or the arch 92 may be substantially planar and/or may be formed within a bending plane. In some embodiments, the outer sheath 82 may include the pre-formed curve and/or the arch 92. In some embodiments, the inner shaft 84 may include the pre-formed curve and/or the arch 92. In some embodiments, both the outer sheath 82 and the inner shaft 84 may include the pre-formed curve and/or the arch 92. In some embodiments, the orientation mandrel 90 may be disposed along the inside bend 94 of the arch 92 of the elongate catheter 80 in a first configuration, as seen in FIG. 4. As shown in FIG. 4, in some embodiments, the orientation lumen 88 and/or the orientation mandrel 90 may be disposed within the bending plane in the first configuration. In some embodiments, the orientation lumen 88 and/or the orientation mandrel 90 may be radially and/or circumferentially offset from the inside bend 94, as seen in FIGS. 4A and 4B. As shown in FIGS. 4A and 4B, in some embodiments, the orientation lumen 88 and/or the orientation mandrel 90 may be disposed outside of (e.g. to one side of) the bending plane in the first configuration.

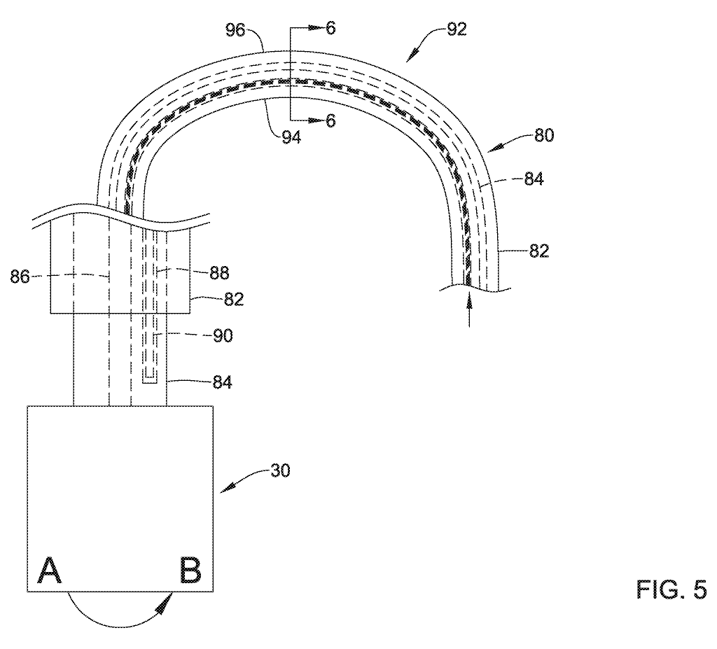

In at least some embodiments, the orientation mechanism 72 may be configured to advance the orientation mandrel 90 distally within the orientation lumen 88 and/or relative to the inner shaft 84, and/or to apply compressive force to the orientation mandrel 90, as seen in FIG. 5. In some embodiments, distal advancement of the distal end of the orientation mandrel 90 and/or applying compressive force against the closed distal end of the orientation lumen 88 may cause compressive force to build up within the orientation mandrel 90. In some embodiments, the compressive force may shift the elongate catheter 80 from the first configuration toward and/or to a second configuration described herein.

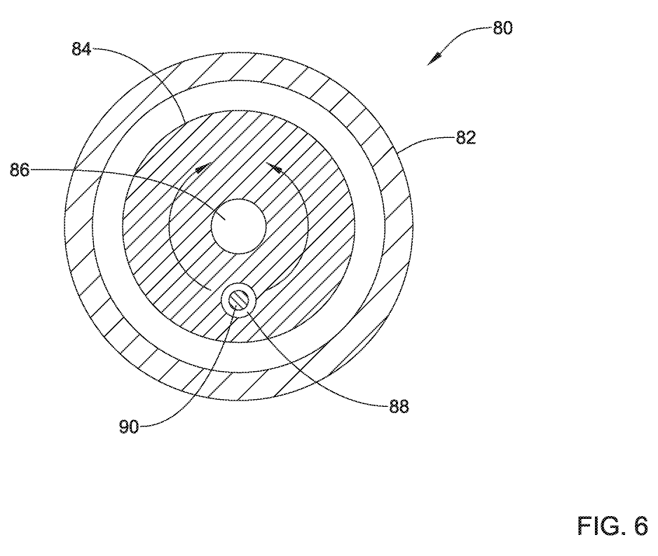

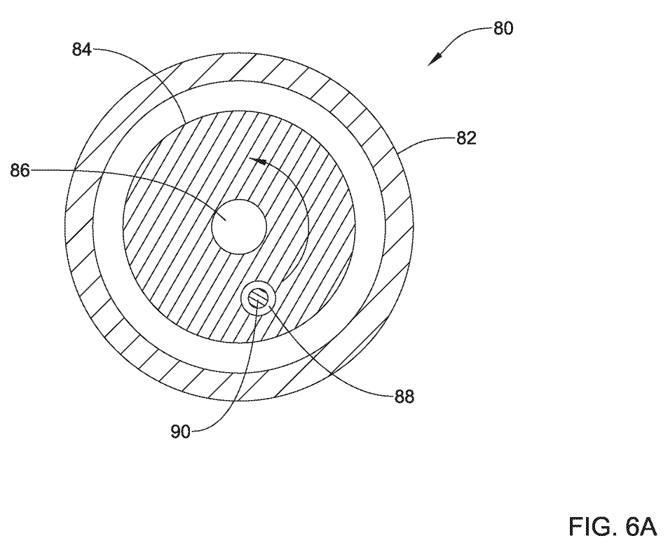

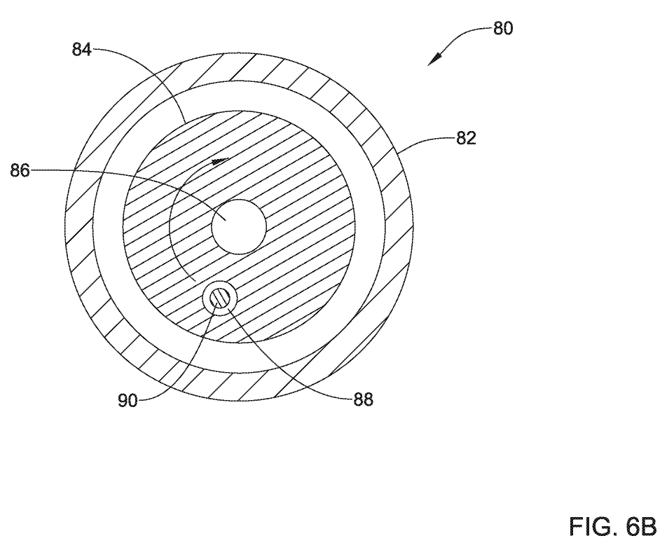

As seen in FIGS. 5 and 6, in some embodiments, the compressive force and/or distal advancement of the distal end of the orientation mandrel 90 against the closed distal end of the orientation lumen 88 may cause the inner shaft 84 to rotate about the central guidewire lumen 86 and/or the central longitudinal axis of the inner shaft 84. Rotation of the inner shaft 84 may cause and/or result in corresponding and/or commensurate rotation of the replacement heart valve implant 30 relative to the outer sheath 82. Accordingly, the orientation mechanism 72 may be used to rotate the inner shaft 84 and/or the replacement heart valve implant 30 as needed to align the commissural posts 42 of the replacement heart valve implant 30 with the commissures 16 of the native heart valve 12 (e.g., the aortic valve).

FIG. 6 illustrates orientation lumen 88 and/or the orientation mandrel 90 disposed within the bending plane in the first configuration. Applying compressive force and/or distal advancement of the distal end of the orientation mandrel 90 against the closed distal end of the orientation lumen 88 when the orientation lumen 88 and/or the orientation mandrel 90 disposed within the bending plane in the first configuration may permit rotation of the inner shaft 84 and/or the replacement heart valve implant 30 toward the second configuration in a first direction or a second direction opposite the first direction.

FIG. 6A illustrates orientation lumen 88 and/or the orientation mandrel 90 disposed outside of (e.g., to one side of; to a first side of; etc.) the bending plane in the first configuration. Applying compressive force and/or distal advancement of the distal end of the orientation mandrel 90 against the closed distal end of the orientation lumen 88 when the orientation lumen 88 and/or the orientation mandrel 90 disposed outside of (e.g., to one side of) the bending plane in the first configuration may permit rotation of the inner shaft 84 and/or the replacement heart valve implant 30 toward the second configuration in a first direction and/or on the first side of the bending plane.

FIG. 6B illustrates orientation lumen 88 and/or the orientation mandrel 90 disposed outside of (e.g., to an opposite side of; to a second side of; etc.) the bending plane in the first configuration. Applying compressive force and/or distal advancement of the distal end of the orientation mandrel 90 against the closed distal end of the orientation lumen 88 when the orientation lumen 88 and/or the orientation mandrel 90 disposed outside of (e.g., to one side of) the bending plane in the first configuration may permit rotation of the inner shaft 84 and/or the replacement heart valve implant 30 toward the second configuration in a second direction opposite the first direction and/or on the second side of the bending plane

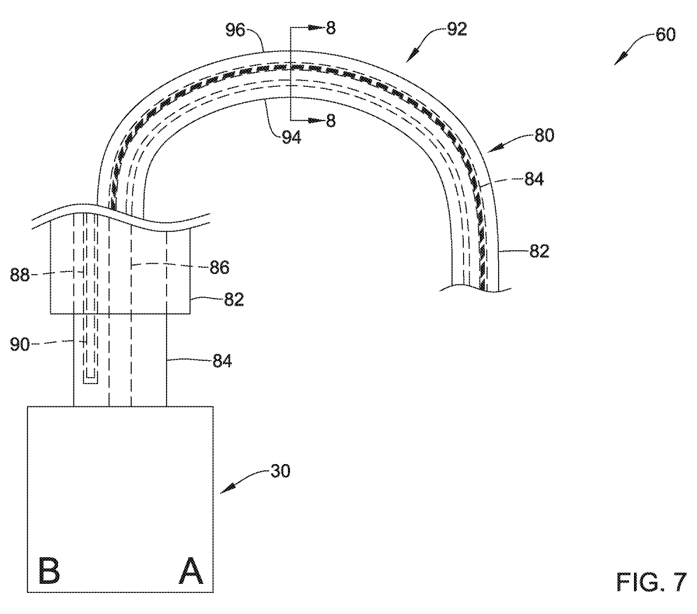

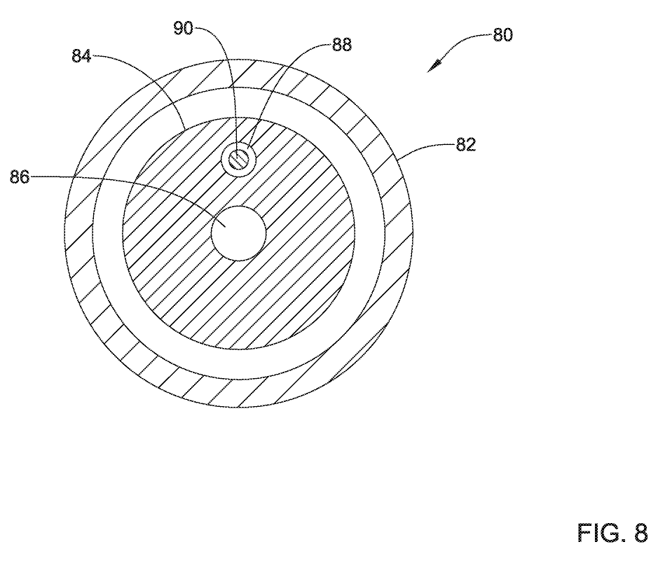

In some embodiments, the orientation mandrel 90 may be moved toward and/or disposed along the outside bend 96 of the arch 92 of the elongate catheter 80 in the second configuration, as seen in FIGS. 7 and 8. As discussed herein, the orientation lumen 88 may be laterally and/or radially offset from the central guidewire lumen 86 and/or the central longitudinal axis of the inner shaft 84, by an offset distance for example. Torque (e.g., rotation) generated within and/or by the inner shaft 84 due to the compressive force and/or distal advancement of the distal end of the orientation mandrel 90 against the closed distal end of the orientation lumen 88 may be greater and/or may increase as the offset distance increases. Accordingly, as the offset distance increases, less axial force and/or axial translation applied to the orientation mandrel 90, and/or less compressive force built up within the orientation mandrel 90, may be necessary to rotate the inner shaft 84 and/or the replacement heart valve implant 30 relative to the outer sheath 82. As may be seen by looking at reference letters A and B in FIG. 7, the replacement heart valve implant 30 has been rotated relative to the outer sheath 82. It will be understood that while some figures illustrate 180 degrees of rotation of the replacement heart valve implant 30, the replacement heart valve implant 30 may be rotate less than 180 degrees as desired. Degrees of rotation may also be affected by placement and/or location of the orientation lumen 88 and/or the orientation mandrel 90 relative to the bending plane. Placement and/or location of the reference letters A and B will be affected accordingly.

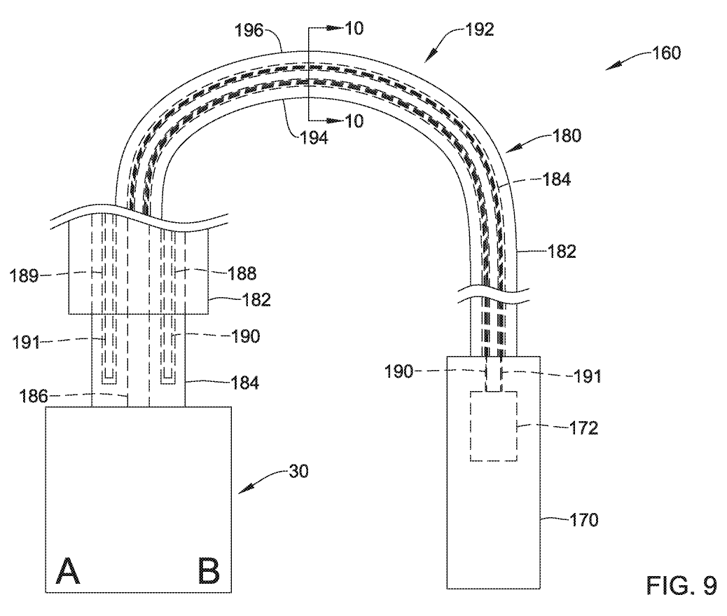

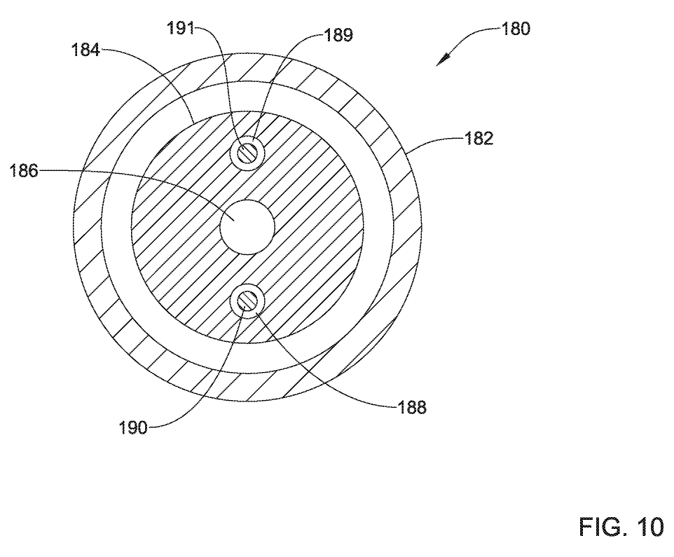

FIGS. 9 and 10 illustrates aspects of an alternative embodiment of the system for delivering the replacement heart valve implant 30 to the native heart valve 12 (e.g., the aortic valve) of the patient. The system may include the replacement heart valve implant 30 as described herein. The system may include a delivery device 160 including a proximal handle 170 and an elongate catheter 180 extending distally from the proximal handle 170. The elongate catheter 180 may include an outer sheath 182 having a lumen extending therethrough and an inner shaft 184 movably disposed within and/or extending longitudinally through the lumen of the outer sheath 182.

In some embodiments, the replacement heart valve implant 30 may be releasably coupled to a distal portion of the inner shaft 184 and disposed within the lumen of the outer sheath 182 in the delivery configuration during delivery of the replacement heart valve implant 30. In some embodiments, the proximal handle 170 may be disposed and/or attached at a proximal end of the delivery device 160 and may include one or more actuation means associated therewith. In some embodiments, the inner shaft 184 may include a central guidewire lumen 186 extending longitudinally therethrough. In some embodiments, the central guidewire lumen 186 may be coaxial with a central longitudinal axis of the inner shaft 184. The central guidewire lumen 186 may be configured to slidably receive and/or slidably move over a guidewire therein. In some embodiments, the inner shaft 184 may include a first orientation lumen 188 extending longitudinally within the inner shaft 184 and a second orientation lumen 189 extending longitudinally within the inner shaft 184. In at least some embodiments, the first orientation lumen 188 and the second orientation lumen 189 may be laterally and/or radially offset from the central guidewire lumen 186 and/or the central longitudinal axis of the inner shaft 184. In some embodiments, the second orientation lumen 189 may be disposed and/or positioned opposite the first orientation lumen 188 relative to the central guidewire lumen 186 and/or the central longitudinal axis of the inner shaft 184. Other configurations are also contemplated. The first orientation lumen 188 may include and/or have a closed distal end. The closed distal end of the first orientation lumen 188 may be disposed proximal of a distal end of the inner shaft 184. The second orientation lumen 189 may include and/or have a closed distal end. The closed distal end of the second orientation lumen 189 may be disposed proximal of the distal end of the inner shaft 184.

In some embodiments, the proximal handle 170 may include an actuation mechanism (not shown) configured to manipulate the position of the outer sheath 182 relative to the inner shaft 184 and/or the replacement heart valve implant 30, and/or to aid in the deployment of the replacement heart valve implant 30. In some embodiments, the actuation mechanism may include a rotatable knob, a slider, or other element. In some embodiments, the delivery device 160 may include a nose cone disposed at a distal end thereof. The delivery device 160 and/or the nose cone may be configured to slidably receive and/or slidably move over the guidewire (not shown). In at least some embodiments, the nose cone may have an atraumatic shape.

During delivery, the replacement heart valve implant 30 may be generally disposed in the delivery configuration within the outer sheath 182 coupled to and/or distal of the inner shaft 184. Once positioned, the outer sheath 182 may be retracted relative to the inner shaft 184 and/or the replacement heart valve implant 30 to expose the replacement heart valve implant 30. The replacement heart valve implant 30 may be actuated using the proximal handle 170 and/or the actuation mechanism in order to translate the replacement heart valve implant 30 into the deployed configuration. When the replacement heart valve implant 30 is suitably deployed within the anatomy, the replacement heart valve implant 30 may be released and/or detached from the delivery device 160 and/or the inner shaft 184 and the delivery device 160 can be removed from the vasculature, leaving the replacement heart valve implant 30 in place within the native heart valve 12 (e.g., the aortic valve) to function as, for example, a suitable replacement for the native heart valve 12.

In some embodiments, the delivery device 160 may include at least one actuator element releasably connecting the replacement heart valve implant 30 to the proximal handle 170 and/or the actuation mechanism. In some embodiments, the actuation mechanism may include an internal system disposed within the proximal handle 170. The internal system may include one or more gears, pulleys, ratchets, screws, cams, or other translation and/or locking mechanisms. In some embodiments, the at least one actuation element may be releasably connected to the commissural posts 42. In some embodiments, the at least one actuation element may be releasably connected to the expandable framework 40. Other configurations are also contemplated. In some embodiments, the at least one actuator element may extend distally from the inner shaft 184 to the replacement heart valve implant 30. In some embodiments, the at least one actuator element may be slidably disposed within and/or may extend slidably through the inner shaft 184 (in one or more unillustrated lumens, for example). In some embodiments, the at least one actuator element may be used to actuate (i.e., translate axially or longitudinally, and/or expand) the replacement heart valve implant 30 between the delivery configuration and the deployed configuration. In some embodiments, the at least one actuator element may include a plurality of actuator elements, two actuator elements, three actuator elements, four actuator elements, or another suitable or desired number of actuator elements.

In some embodiments, the delivery device 160 may include a first orientation mandrel 190 slidably disposed within the first orientation lumen 188. The first orientation mandrel 190 may extend distally from the proximal handle 170 within the first orientation lumen 188 to a distal end disposed and/or positioned proximate the closed distal end of the first orientation lumen 188. The first orientation mandrel 190 may be operably coupled to an orientation mechanism 172 of the proximal handle 170. The orientation mechanism 172 may be configured to move at least a portion of the first orientation mandrel 190 axially and/or longitudinally within the first orientation lumen 188 and/or relative to the inner shaft 184.

In some embodiments, the delivery device 160 may include a second orientation mandrel 191 slidably disposed within the second orientation lumen 189. The second orientation mandrel 191 may extend distally from the proximal handle 170 within the second orientation lumen 189 to a distal end disposed and/or positioned proximate the closed distal end of the second orientation lumen 189. The second orientation mandrel 191 may be operably coupled to the orientation mechanism 172 of the proximal handle 170. The orientation mechanism 172 may be configured to move at least a portion of the second orientation mandrel 191 axially and/or longitudinally within the second orientation lumen 189 and/or relative to the inner shaft 184.

In some embodiments, the orientation mechanism 172 may include an internal system disposed within the proximal handle 170. The internal system may include one or more gears, pulleys, ratchets, cams, screws, or other translation and/or locking mechanisms capable of imparting linear movement, compressive force, and/or tensile force upon the first orientation mandrel 190 and/or the second orientation mandrel 191.

In some embodiments, a distal portion of the elongate catheter 180 may include a pre-formed curve configured to conform to the aortic arch 22 (e.g., FIG. 1) of the patient. In some embodiments, the pre-formed curve may define an arch 192 of the elongate catheter 180 having an inside bend 194 and an outside bend 196 disposed opposite the inside bend 194 relative to the inner shaft 184 and/or the central longitudinal axis of the inner shaft 184. In some embodiments, the pre-formed curve and/or the arch 192 may be substantially planar and/or may be formed within a bending plane. In some embodiments, the outer sheath 182 may include the pre-formed curve and/or the arch 192. In some embodiments, the inner shaft 184 may include the pre-formed curve and/or the arch 192. In some embodiments, both the outer sheath 182 and the inner shaft 184 may include the pre-formed curve and/or the arch 192. In some embodiments, the first orientation mandrel 190 may be disposed along the inside bend 194 of the arch 192 of the elongate catheter 180 and the second orientation mandrel 191 may be disposed along the outside bend 196 of the arch 192 of the elongate catheter 180 in a first configuration, as seen in FIG. 10.

As shown in FIG. 10, in some embodiments, the first orientation lumen 188 and/or the first orientation mandrel 190 may be disposed within the bending plane in the first configuration. In some embodiments, the second orientation lumen 189 and/or the second orientation mandrel 191 may be disposed within the bending plane in the first configuration. In some embodiments, the first orientation lumen 188 and/or the first orientation mandrel 190 and the second orientation lumen 189 and/or the second orientation mandrel 191 may be radially and/or circumferentially offset from the inside bend 194, similar to the configuration shown in FIGS. 4A and 4B. In some embodiments, the first orientation lumen 188 and/or the first orientation mandrel 190 and the second orientation lumen 189 and/or the second orientation mandrel 191 may be disposed outside of (e.g. to one side of) the bending plane in the first configuration.

In some embodiments, the orientation mechanism 172 may be configured to advance the first orientation mandrel 190 distally within the first orientation lumen 188 and/or relative to the inner shaft 184. In some embodiments, distal advancement of the distal end of the first orientation mandrel 190 against the closed distal end of the first orientation lumen 188 may cause compressive force to build up within the first orientation mandrel 190. In some embodiments, the compressive force may shift the elongate catheter 180 from the first configuration toward and/or to a second configuration described herein.

In some embodiments, the compressive force and/or distal advancement of the distal end of the first orientation mandrel 190 against the closed distal end of the first orientation lumen 188 may cause the inner shaft 184 to rotate about the central guidewire lumen 186 and/or the central longitudinal axis of the inner shaft 184 in a first direction. Rotation of the inner shaft 184 in the first direction may cause and/or result in corresponding and/or commensurate rotation of the replacement heart valve implant 30 relative to the outer sheath 182 in the first direction.

In some embodiments, the orientation mechanism 172 may be configured to advance the second orientation mandrel 191 distally within the second orientation lumen 189 and/or relative to the inner shaft 184. In some embodiments, distal advancement of the distal end of the second orientation mandrel 191 against the closed distal end of the second orientation lumen 189 may cause compressive force to build up within the second orientation mandrel 191. In some embodiments, the compressive force may shift the elongate catheter 180 from the first configuration toward and/or to a second configuration described herein.

In some embodiments, the compressive force and/or distal advancement of the distal end of the second orientation mandrel 191 against the closed distal end of the second orientation lumen 189 may cause the inner shaft 184 to rotate about the central guidewire lumen 186 and/or the central longitudinal axis of the inner shaft 184 in a second direction opposite the first direction. Rotation of the inner shaft 184 in the second direction may cause and/or result in corresponding and/or commensurate rotation of the replacement heart valve implant 30 relative to the outer sheath 182 in the second direction. Accordingly, the orientation mechanism 172 may be used to rotate the inner shaft 184 and/or the replacement heart valve implant 30 in the first direction and/or the second direction as needed to align the commissural posts 42 of the replacement heart valve implant 30 with the commissures 16 of the native heart valve 12 (e.g., the aortic valve). In some embodiments, the first orientation mandrel 190 may be moved toward and/or disposed along the outside bend 196 of the arch 192 of the elongate catheter 180 and the second orientation mandrel 191 may be moved toward and/or disposed along the inside bend 194 of the arch 192 of the elongate catheter 180 in the second configuration.

As discussed herein, the first orientation lumen 188 and the second orientation lumen 189 may be laterally and/or radially offset from the central guidewire lumen 186 and/or the central longitudinal axis of the inner shaft 184, by an offset distance for example. Torque (e.g., rotation) generated within and/or by the inner shaft 184 due to the compressive force and/or distal advancement of the distal end of the first orientation mandrel 190 against the closed distal end of the first orientation lumen 188 and/or the second orientation mandrel 191 against the closed distal end of the second orientation lumen 189 may be greater and/or may increase as the offset distance increases. Accordingly, as the offset distance increases, less axial force and/or axial translation applied to the first orientation mandrel 190 and/or the second orientation mandrel 191, and/or less compressive force built up within the first orientation mandrel 190 and/or the second orientation mandrel 191, may be necessary to rotate the inner shaft 184 and/or the replacement heart valve implant 30 relative to the outer sheath 182.

In an alternative configuration, the distal end of the first orientation mandrel 190 and/or the second orientation mandrel 191 may be fixedly secured to the inner shaft 184 and/or within the first orientation lumen 188 and/or the second orientation lumen 189, respectively. Tensile force may be applied to the first orientation mandrel 190 and/or the second orientation mandrel 191 using and/or by the orientation mechanism 172 to reverse the direction of rotation of the inner shaft 184 and/or the replacement heart valve implant 30 relative to the outer sheath 182.

In some embodiments, directional control over rotation of the inner shaft 184 and/or the replacement heart valve implant 30 relative to the outer sheath 182 may be provided by locating the first orientation lumen 188 and/or the first orientation mandrel 190, and the second orientation lumen 189 and/or the second orientation mandrel 191, outside of (e.g., to one side of ) the bending plane in the first configuration, as described above with respect to FIGS. 4-6. Applying compressive force and/or tensile force to the first orientation mandrel 190 may cause rotation of the inner shaft 184 and/or the replacement heart valve implant 30 relative to the outer sheath 182 along and/or on a first side of the bending plane. Applying compressive force and/or tensile force to the second orientation mandrel 191 may cause rotation of the inner shaft 184 and/or the replacement heart valve implant 30 relative to the outer sheath 182 along and/or on a second side of the bending plane opposite the first side of the bending plane.

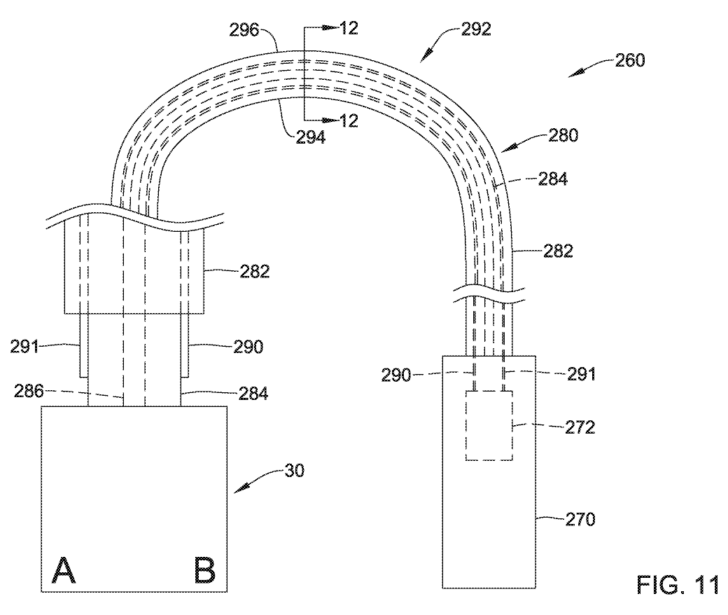

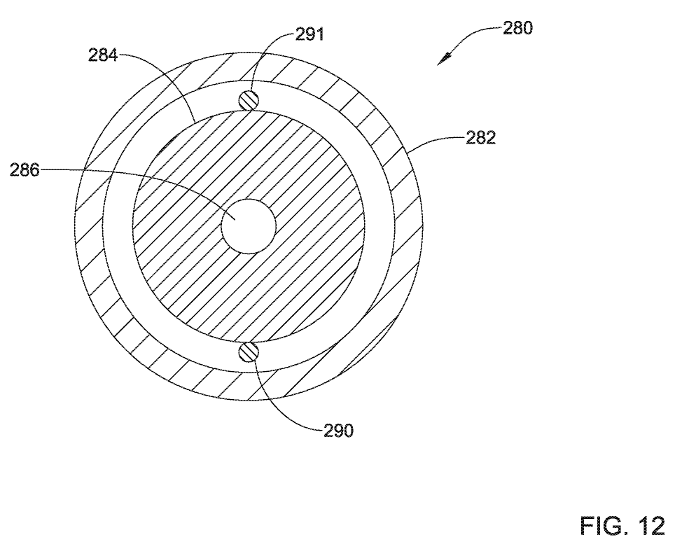

FIGS. 11 and 12 illustrates aspects of an alternative embodiment of a system for delivering the replacement heart valve implant 30 to the native heart valve 12 (e.g., the aortic valve) of the patient. The system may include the replacement heart valve implant 30 as described herein. The system may include a delivery device 260 including a proximal handle 270 and an elongate catheter 280 extending distally from the proximal handle 270. The elongate catheter 280 may include an outer sheath 282 having a lumen extending therethrough and an inner shaft 284 movably disposed within and/or extending longitudinally through the lumen of the outer sheath 282.

In some embodiments, the replacement heart valve implant 30 may be releasably coupled to a distal portion of the inner shaft 284 and disposed within the lumen of the outer sheath 282 in the delivery configuration during delivery of the replacement heart valve implant 30. In some embodiments, the proximal handle 270 may be disposed and/or attached at a proximal end of the delivery device 260 and may include one or more actuation means associated therewith. In some embodiments, the inner shaft 284 may include a central guidewire lumen 286 extending longitudinally therethrough. In some embodiments, the central guidewire lumen 286 may be coaxial with a central longitudinal axis of the inner shaft 284. The central guidewire lumen 286 may be configured to slidably receive and/or slidably move over a guidewire therein.

In some embodiments, the proximal handle 270 may include an actuation mechanism (not shown) configured to manipulate the position of the outer sheath 282 relative to the inner shaft 284 and/or the replacement heart valve implant 30, and/or to aid in the deployment of the replacement heart valve implant 30. In some embodiments, the actuation mechanism may include a rotatable knob, a slider, or other element. In some embodiments, the delivery device 260 may include a nose cone disposed at a distal end thereof. The delivery device 260 and/or the nose cone may be configured to slidably receive and/or slidably move over the guidewire (not shown). In at least some embodiments, the nose cone may have an atraumatic shape.

During delivery, the replacement heart valve implant 30 may be generally disposed in the delivery configuration within the outer sheath 282 coupled to and/or distal of the inner shaft 284. Once positioned, the outer sheath 282 may be retracted relative to the inner shaft 284 and/or the replacement heart valve implant 30 to expose the replacement heart valve implant 30. The replacement heart valve implant 30 may be actuated using the proximal handle 270 and/or the actuation mechanism in order to translate the replacement heart valve implant 30 into the deployed configuration. When the replacement heart valve implant 30 is suitably deployed within the anatomy, the replacement heart valve implant 30 may be released and/or detached from the delivery device 260 and/or the inner shaft 284 and the delivery device 260 can be removed from the vasculature, leaving the replacement heart valve implant 30 in place within the native heart valve 12 (e.g., the aortic valve) to function as, for example, a suitable replacement for the native heart valve 12.

In some embodiments, the delivery device 260 may include at least one actuator element releasably connecting the replacement heart valve implant 30 to the proximal handle 270 and/or the actuation mechanism. In some embodiments, the actuation mechanism may include an internal system disposed within the proximal handle 270. The internal system may include one or more gears, pulleys, ratchets, screws, cams, or other translation and/or locking mechanisms. In some embodiments, the at least one actuation element may be releasably connected to the commissural posts 42. In some embodiments, the at least one actuation element may be releasably connected to the expandable framework 40. Other configurations are also contemplated. In some embodiments, the at least one actuator element may extend distally from the inner shaft 284 to the replacement heart valve implant 30. In some embodiments, the at least one actuator element may be slidably disposed within and/or may extend slidably through the inner shaft 284 (in one or more unillustrated lumens, for example). In some embodiments, the at least one actuator element may be used to actuate (i.e., translate axially or longitudinally, and/or expand) the replacement heart valve implant 30 between the delivery configuration and the deployed configuration. In some embodiments, the at least one actuator element may include a plurality of actuator elements, two actuator elements, three actuator elements, four actuator elements, or another suitable or desired number of actuator elements.

In some embodiments, the delivery device 260 may include a first orientation mandrel 290 attached to an outside surface of the inner shaft 284. The first orientation mandrel 290 may extend distally from the proximal handle 270 to a first distal end fixedly attached to the inner shaft 284 proximate a distal end of the inner shaft 284. The first orientation mandrel 290 may be operably coupled to an orientation mechanism 272 of the proximal handle 270. The orientation mechanism 272 may be configured to move at least a portion of the first orientation mandrel 290 axially and/or longitudinally relative to the inner shaft 284.

In some embodiments, the delivery device 260 may include a second orientation mandrel 291 attached to the outside surface of the inner shaft 284 opposite the first orientation mandrel 290 relative to the central guidewire lumen 286 and/or the central longitudinal axis of the inner shaft 284. The second orientation mandrel 291 may extend distally from the proximal handle 270 to a second distal end fixedly attached to the inner shaft 284 proximate the distal end of the inner shaft 284. The second orientation mandrel 291 may be operably coupled to the orientation mechanism 272 of the proximal handle 270. The orientation mechanism 272 may be configured to move at least a portion of the second orientation mandrel 291 axially and/or longitudinally relative to the inner shaft 284.

In some embodiments, the orientation mechanism 272 may include an internal system disposed within the proximal handle 270. The internal system may include one or more gears, pulleys, ratchets, cams, screws, or other translation and/or locking mechanisms capable of imparting linear movement, compressive force, and/or tensile force upon the first orientation mandrel 290 and/or the second orientation mandrel 291.

In some embodiments, a distal portion of the elongate catheter 280 may include a pre-formed curve configured to conform to the aortic arch 22 (e.g., FIG. 1) of the patient. In some embodiments, the pre-formed curve may define an arch 292 of the elongate catheter 280 having an inside bend 294 and an outside bend 296 disposed opposite the inside bend 294 relative to the inner shaft 284 and/or the central longitudinal axis of the inner shaft 284. In some embodiments, the pre-formed curve and/or the arch 292 may be substantially planar and/or may be formed within a bending plane. In some embodiments, the outer sheath 282 may include the pre-formed curve and/or the arch 292. In some embodiments, the inner shaft 284 may include the pre-formed curve and/or the arch 292. In some embodiments, both the outer sheath 282 and the inner shaft 284 may include the pre-formed curve and/or the arch 292. In some embodiments, the first orientation mandrel 290 may be disposed along the inside bend 294 of the arch 292 of the elongate catheter 280 and the second orientation mandrel 291 may be disposed along the outside bend 296 of the arch 292 of the elongate catheter 280 in a first configuration, as seen in FIG. 12.

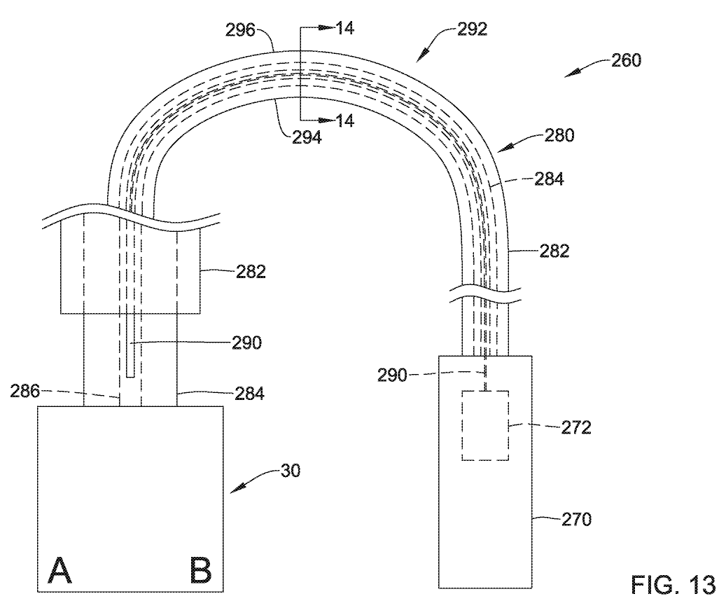

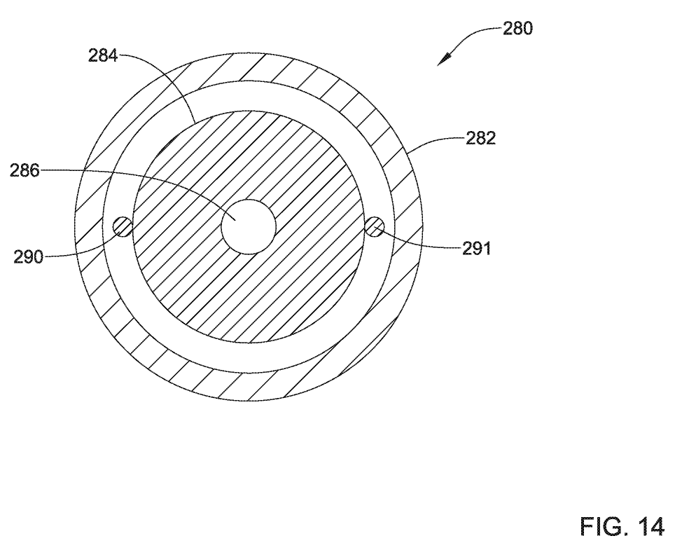

As shown in FIG. 12, in some embodiments, the first orientation mandrel 290 and the second orientation mandrel 291 may be disposed within the bending plane in the first configuration. In some embodiments, the first orientation mandrel 290 and the second orientation mandrel 291 may be radially and/or circumferentially offset from the inside bend 294, as seen in FIGS. 13 and 14. In some embodiments, the first orientation mandrel 190 and the second orientation mandrel 191 may be disposed outside of (e.g. to one side of) the bending plane in the first configuration. In some embodiments, the first orientation mandrel 290 and the second orientation mandrel 291 may be positioned to define a preferred bending plane that is independent of and/or such that the distal portion of the elongate catheter 280 does not include the pre-formed curve. The preferred bending plane may conform to and/or self-align with the aortic arch 22 when the elongate catheter 280 is positioned therein. In some embodiments, the first orientation mandrel 290 and the second orientation mandrel 291 may be positioned opposite each other relative to the preferred bending plane. In some embodiments, the first orientation mandrel 290 and the second orientation mandrel 291 may be positioned at approximately 90 degrees to the preferred bending plane, as shown in FIGS. 13 and 14.

In some embodiments, the orientation mechanism 272 may be configured to advance at least a portion of the first orientation mandrel 290 distally relative to the inner shaft 284. In some embodiments, distal advancement against the first distal end of the first orientation mandrel 290 fixedly attached to the inner shaft 284 may cause compressive force to build up within the first orientation mandrel 290. In some embodiments, the compressive force may shift the elongate catheter 280 from the first configuration toward and/or to a second configuration described herein.

In some embodiments, the compressive force and/or distal advancement of at least a portion of the first orientation mandrel 290 against the first distal end of the first orientation mandrel 290 fixedly attached to the inner shaft 284 may cause the inner shaft 284 to rotate about the central guidewire lumen 286 and/or the central longitudinal axis of the inner shaft 284 in a first direction to shift the elongate catheter 280 from the first configuration toward and/or to the second configuration. Rotation of the inner shaft 284 in the first direction may cause and/or result in corresponding and/or commensurate rotation of the replacement heart valve implant 30 relative to the outer sheath 282 in the first direction. In some embodiments, the first direction is counterclockwise as viewed distally to proximally along the inner shaft 284.

In some embodiments, the orientation mechanism 272 may be configured to retract at least a portion of the first orientation mandrel 290 proximally relative to the inner shaft 284. In some embodiments, proximal retraction of the first distal end of the first orientation mandrel 290 fixedly attached to the inner shaft 284 may cause a tensile force to build up within the first orientation mandrel 290. In some embodiments, the tensile force may shift the elongate catheter 280 from the first configuration toward and/or to the second configuration described herein.

In some embodiments, the tensile force and/or proximal retraction of the first distal end of the first orientation mandrel 290 fixedly attached to the inner shaft 284 may cause the inner shaft 284 to rotate about the central guidewire lumen 286 and/or the central longitudinal axis of the inner shaft 284 in a second direction opposite the first direction to shift the elongate catheter 280 from the first configuration toward and/or to the second configuration. Rotation of the inner shaft 284 in the second direction may cause and/or result in corresponding and/or commensurate rotation of the replacement heart valve implant 30 relative to the outer sheath 282 in the second direction. In some embodiments, the second direction is clockwise as viewed distally to proximally along the inner shaft 284.

In some embodiments, the orientation mechanism 272 may be configured to advance at least a portion of the second orientation mandrel 291 distally relative to the inner shaft 184. In some embodiments, distal advancement against the second distal end of the second orientation mandrel 291 fixedly attached to the inner shaft 284 may cause compressive force to build up within the second orientation mandrel 291. In some embodiments, the compressive force may shift the elongate catheter 280 from the first configuration toward and/or to a second configuration described herein.

In some embodiments, the compressive force and/or distal advancement of at least a portion of the second orientation mandrel 291 against the second distal end of the second orientation mandrel 291 fixedly attached to the inner shaft 284 may cause the inner shaft 284 to rotate about the central guidewire lumen 286 and/or the central longitudinal axis of the inner shaft 284 in the second direction opposite the first direction to shift the elongate catheter 280 from the first configuration toward and/or to the second configuration. Rotation of the inner shaft 284 in the second direction may cause and/or result in corresponding and/or commensurate rotation of the replacement heart valve implant 30 relative to the outer sheath 282 in the second direction. In some embodiments, the second direction is clockwise as viewed distally to proximally along the inner shaft 284.

Accordingly, the orientation mechanism 272 may be used to rotate the inner shaft 284 and/or the replacement heart valve implant 30 in the first direction and/or the second direction as needed to align the commissural posts 42 of the replacement heart valve implant 30 with the commissures 16 of the native heart valve 12 (e.g., the aortic valve). In some embodiments, the first orientation mandrel 290 may be moved toward and/or disposed along the outside bend 296 of the arch 292 of the elongate catheter 280 and the second orientation mandrel 291 may be moved toward and/or disposed along the inside bend 294 of the arch 292 of the elongate catheter 280 in the second configuration.

In some embodiments, directional control over rotation of the inner shaft 284 and/or the replacement heart valve implant 30 relative to the outer sheath 282 may be provided by locating the first orientation mandrel 290 and the second orientation mandrel 191 outside of (e.g., to one side of ) the bending plane and/or the preferred bending plane in the first configuration, as described above with respect to FIGS. 4-6. For example, applying compressive force and/or tensile force to the first orientation mandrel 290 may cause rotation of the inner shaft 284 and/or the replacement heart valve implant 30 relative to the outer sheath 282 along and/or on a first side of the bending plane and/or the preferred bending plane. Applying compressive force and/or tensile force to the second orientation mandrel 291 may cause rotation of the inner shaft 284 and/or the replacement heart valve implant 30 relative to the outer sheath 282 along and/or on a second side of the bending plane and/or the preferred bending plane opposite the first side of the bending plane and/or the preferred bending plane.

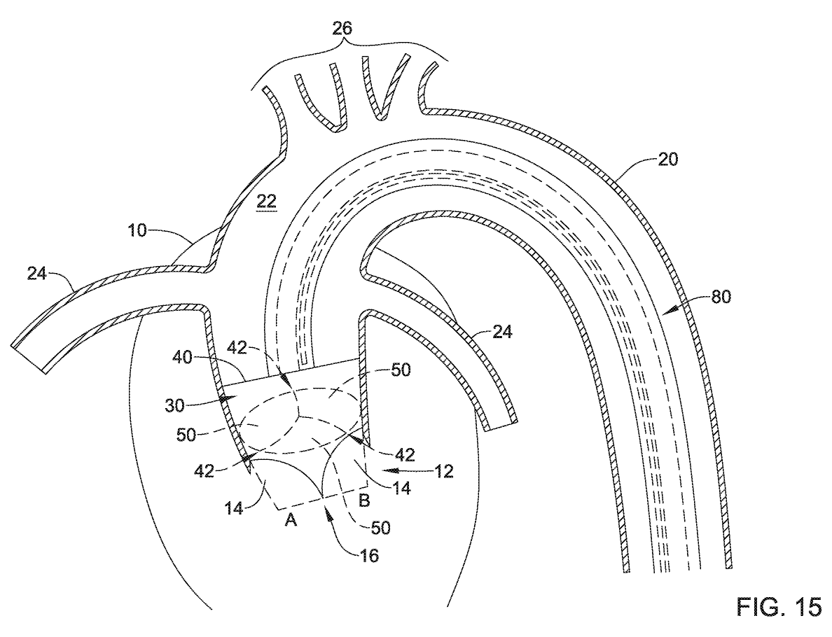

FIGS. 15-17 illustrates aspects of a method of positioning the replacement heart valve implant 30 within the native heart valve 12 (e.g., the aortic valve) of the patient. While discussed in the context of the system of FIGS. 3-8, it will be appreciated that the method may also apply to the systems of FIGS. 9-14 as described herein.

The method may include advancing the system percutaneously to a position adjacent the native heart valve 12 (e.g., the aortic valve). The system may include and/or comprise structures, elements, and/or features as described herein. As seen in FIG. 15, the elongate catheter 80 may extend and/or be advanced within the aorta 20 and through the aortic arch 22. The replacement heart valve implant 30 may be advanced to the position adjacent the native heart valve 12 within the outer sheath 82 in the delivery configuration.

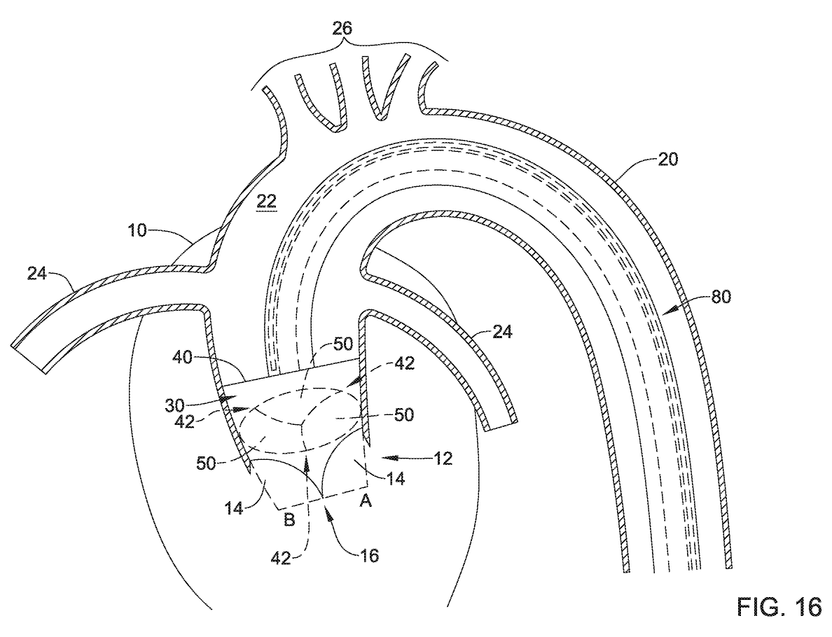

After reaching the position adjacent the native heart valve 12, the method may include deploying the replacement heart valve implant 30 within the native heart valve 12. As may be seen in FIG. 15, in some embodiments, upon initial deployment, the commissural posts 42 of the replacement heart valve implant 30 may be rotationally misaligned with and/or rotationally offset from the commissures 16 of the native heart valve 12. As such, the method may include aligning the commissural posts 42 of the replacement heart valve implant 30 with the commissures 16 of the native heart valve 12 by rotating the replacement heart valve implant 30 relative to the outer sheath 82 of the elongate catheter 80, as shown in FIG. 16.

In some embodiments, rotational alignment of the commissural posts 42 of the replacement heart valve implant 30 with the commissures 16 of the native heart valve 12 may be useful for reducing stress on the plurality of leaflets 50 and/or increasing/maximizing the life of the replacement heart valve implant 30. In some embodiments, rotation of the replacement heart valve implant 30 may be useful for preserving access to the coronary arteries 24.

In previous TAVI systems, rotation of the entire system is required in order to rotate the replacement heart valve implant relative to the native heart valve due to construction of the system, interaction of various components of the system, etc. This may be very difficult to achieve accurately as a result of the length of the delivery device, the flexibility of the delivery device, and/or the tortuous anatomy the delivery device was navigated through to reach the patient’s heart, for example. The disclosed systems and/or method(s) may permit fine tuning of the rotational alignment of the replacement heart valve implant 30 relative to the native heart valve 12 without needing to rotate the entire system, thus making rotation of the replacement heart valve implant 30 more precise, reducing the forces needed to rotate the replacement heart valve implant 30, and improving patient safety and/or comfort.

In some embodiments, the method may include advancing the orientation mandrel 90 axially in a distal direction using the orientation mechanism 72 to rotate the inner shaft 84 and/or the replacement heart valve implant 30 relative to the outer sheath 82. In some embodiments, the method may include advancing the orientation mandrel 90 axially in the distal direction within the orientation lumen 88 and/or relative to the inner shaft 84 to rotate the inner shaft 84 and/or the replacement heart valve implant 30 relative to the outer sheath 82. As discussed herein, the reference letters A and B in FIGS. 15 and 16 indicate that the replacement heart valve implant 30 was rotated about 180 degrees. In some embodiments, the replacement heart valve implant 30 may be rotated less than 180 degrees. For example, in some embodiments, rotation of about 60 degrees or less than 60 degrees may be sufficient to align the commissural posts 42 of the replacement heart valve implant 30 with the commissures 16 of the native heart valve 12. In some embodiments, more or less rotation may be needed or desired to facilitate preservation of access to the coronary arteries 24, for example, depending on features found in the replacement heart valve implant 30 and/or the expandable framework 40.

In some embodiments, prior to advancing the orientation mandrel 90 axially in the distal direction, the orientation mandrel 90 may be disposed along the inside bend 94 of the arch 92 of the elongate catheter 80 within the aortic arch 22 of the patient, as seen in FIG. 15. In some embodiments, after advancing the orientation mandrel 90 axially in the distal direction, the orientation mandrel 90 may be disposed along the outside bend 96 of the arch 92 of the elongate catheter 80 within the aortic arch 22 of the patient, as seen in FIG. 16. As discussed herein, other configurations and/or positioning of the orientation mandrel 90 with respect to the arch 92 and/or the central longitudinal axis of the inner shaft 84 are also contemplated.

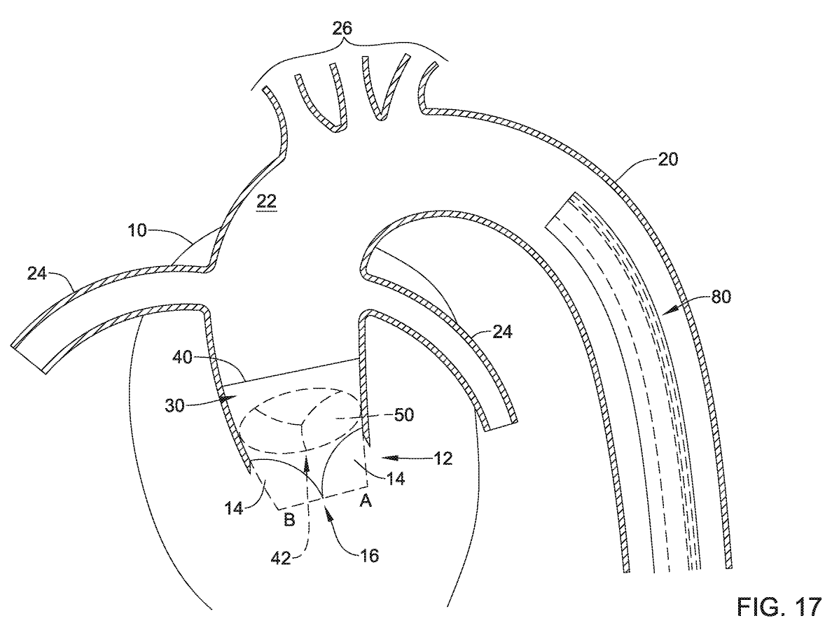

In some embodiments, the method may further include releasing the replacement heart valve implant 30 within the native heart valve 12 of the patient. The expandable framework 40 may be in the deployed configuration with the replacement heart valve implant 30 is released within the native heart valve 12 of the patient, as seen in FIG. 17. The method may further include withdrawing the elongate catheter 80 of the delivery device 60 (not shown) proximally and removing the elongate catheter 80 from the patient.

The materials that can be used for the various components of the system (and/or other elements disclosed herein) and the various components thereof disclosed herein may include those commonly associated with medical devices and/or systems. For simplicity purposes, the following discussion makes reference to the system. However, this is not intended to limit the devices and methods described herein, as the discussion may be applied to other elements, members, components, or devices disclosed herein, such as, but not limited to, the replacement heart valve implant, the elongate catheter, the outer sheath, the inner shaft, the handle, the delivery device, etc. and/or elements or components thereof.

In some embodiments, the system and/or other elements disclosed herein may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated "linear elastic" or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial "superelastic plateau" or "flag region" in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about –60 degrees Celsius (ºC) to about 120 ºC in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the system and/or other elements disclosed herein may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the system and/or other elements disclosed herein. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system and/or other elements disclosed herein to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the system and/or other elements disclosed herein may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni-Co-Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps, without exceeding the scope of the disclosure. This may include, to the extent that it is appropriate, the use of any of the features of one example embodiment being used in other embodiments. The disclosure’s scope is, of course, defined in the language in which the appended claims are expressed.

What is claimed is:

1. A method of delivering and positioning a replacement heart valve implant within a native heart valve of a patient, comprising:
advancing a delivery system percutaneously through vasculature to a position adjacent the native heart valve, the delivery system comprising:
a replacement heart valve implant including an expandable framework and a plurality of leaflets coupled to the expandable framework at commissural posts of the expandable framework; and
a delivery device including a proximal handle and an elongate catheter extending distally from the proximal handle, wherein the elongate catheter includes an outer sheath and an inner shaft movably disposed within a lumen of the outer sheath,
wherein the replacement heart valve implant is releasably coupled to a distal portion of the inner shaft in a delivery configuration;
wherein the inner shaft includes a central guidewire lumen extending longitudinally therethrough;
wherein the inner shaft includes an orientation lumen extending longitudinally within the inner shaft, the orientation lumen being laterally offset from the central guidewire lumen and having a closed distal end;
wherein the delivery device includes an orientation mandrel slidably disposed within the orientation lumen;
positioning the elongate catheter such that a distal portion thereof conforms to an aortic arch of the patient;
deploying the replacement heart valve implant within the native heart valve; and
aligning the commissural posts of the replacement heart valve implant with commissures of the native heart valve by actuating the orientation mandrel to rotate the replacement heart valve implant relative to the outer sheath.

2. The method of claim 1, wherein the orientation mandrel extends distally from the proximal handle within the orientation lumen to a distal end proximate the closed distal end of the orientation lumen.

3. The method of claim 2, wherein the orientation mandrel is operably coupled to an orientation mechanism of the proximal handle, and wherein actuating the orientation mandrel comprises operating the orientation mechanism to move the orientation mandrel axially relative to the inner shaft.

4. The method of claim 3, wherein the distal portion of the elongate catheter includes a pre-formed curve defining an arch of the elongate catheter having an inside bend and an outside bend disposed opposite the inside bend relative to the central guidewire lumen.

5. The method of claim 4, wherein prior to actuating the orientation mandrel, the orientation mandrel is disposed along the inside bend of the arch of the elongate catheter within the aortic arch of the patient.

6. The method of claim 5, wherein actuating the orientation mandrel comprises advancing the orientation mandrel distally relative to the inner shaft.

7. The method of claim 6, wherein advancing the orientation mandrel distally causes the distal end of the orientation mandrel to contact the closed distal end of the orientation lumen, thereby building up compressive force within the orientation mandrel.

8. The method of claim 7, wherein the compressive force causes the orientation mandrel to shift from being disposed along the inside bend to being disposed along the outside bend of the arch of the elongate catheter.

9. A method of rotationally aligning a replacement heart valve implant within a native heart valve of a patient, comprising:
percutaneously advancing a delivery system to a target location adjacent the native heart valve, the delivery system comprising:
a replacement heart valve implant including an expandable framework and a plurality of leaflets coupled to the expandable framework at commissural posts of the expandable framework; and
a delivery device including a proximal handle and an elongate catheter extending distally from the proximal handle, wherein the elongate catheter includes an outer sheath and an inner shaft movably disposed within a lumen of the outer sheath;
wherein the replacement heart valve implant is releasably coupled to a distal portion of the inner shaft in a delivery configuration;
wherein the inner shaft includes a central guidewire lumen extending longitudinally therethrough;
wherein the delivery device includes a first orientation mandrel attached to an outside surface of the inner shaft;
deploying the replacement heart valve implant within the native heart valve;
determining rotational alignment between the commissural posts of the replacement heart valve implant and commissures of the native heart valve; and
adjusting rotational alignment by applying force to the first orientation mandrel to rotate the replacement heart valve implant relative to the outer sheath.

10. The method of claim 9, wherein the first orientation mandrel extends distally from the proximal handle to a first distal end fixedly attached to the inner shaft proximate a distal end of the inner shaft.

11. The method of claim 10, wherein the first orientation mandrel is operably coupled to an orientation mechanism of the proximal handle, and wherein applying force comprises operating the orientation mechanism to apply compressive force or tensile force to the first orientation mandrel.

12. The method of claim 11, wherein a distal portion of the elongate catheter includes a pre-formed curve configured to conform to an aortic arch of the patient, the pre-formed curve defining an arch having an inside bend and an outside bend.

13. The method of claim 12, wherein prior to adjusting rotational alignment, the first orientation mandrel is disposed along the inside bend of the arch of the elongate catheter.

14. The method of claim 13, wherein applying compressive force against the first distal end causes rotation of the inner shaft in a first direction.

15. The method of claim 13, wherein applying tensile force to the first distal end causes rotation of the inner shaft in a second direction opposite the first direction.

16. The method of claim 14, wherein the delivery device includes a second orientation mandrel attached to an outside surface of the inner shaft opposite the first orientation mandrel relative to the central guidewire lumen.

17. The method of claim 16, further comprising selectively applying compressive force or tensile force to the second orientation mandrel to achieve bidirectional rotational control of the replacement heart valve implant.

18. A method of precisely controlling rotational orientation of a replacement heart valve implant during deployment, comprising:
providing a delivery system including a replacement heart valve implant releasably coupled to an inner shaft of a delivery device, wherein the inner shaft includes at least one orientation element configured to impart rotational movement;
navigating the delivery system through vasculature to position the replacement heart valve implant adjacent a native heart valve;
initiating deployment of the replacement heart valve implant from a delivery configuration toward a deployed configuration;
monitoring rotational alignment between commissural posts of the replacement heart valve implant and commissures of the native heart valve;
actuating the at least one orientation element to make fine rotational adjustments to the replacement heart valve implant without rotating an entirety of the delivery system; and
completing deployment of the replacement heart valve implant in proper rotational alignment with the native heart valve.

19. The method of claim 18, wherein the at least one orientation element comprises an orientation mandrel disposed within an orientation lumen of the inner shaft, and wherein actuating comprises applying axial force to the orientation mandrel.

20. The method of claim 18, wherein the at least one orientation element comprises at least one orientation mandrel attached to an exterior surface of the inner shaft, and wherein actuating comprises applying compressive or tensile force to the at least one orientation mandrel.
